(19) United States
(12) Patent Application Publication
(10) Pub. No.: US 2011/0197886 A1
(43) Pub. Date: Aug. 18, 2011

(54) RESPIRATORY DEVICE AND METHOD FOR VENTILATING A PATIENT

(76) Inventors: Josef Guttmann, Freiburg-Munzingen (DE); Claudius Stahl, Heuweiler (DE); Knut Möller, Kirchzarten (DE)

(21) Appl. No.: 11/578,590

(22) PCT Filed: Apr. 13, 2005
(86) PCT No.: PCT/EP2005/003858

§ 371 Date: Oct. 5, 2007

(30) Foreign Application Priority Data
Apr. 16, 2004 (DE) 10 2004 019 122.0

(51) Int. Cl. A61M 16/00

(52) U.S. Cl. 128/204.23

(57) ABSTRACT

The invention relates to a respiratory device for ventilating a patient. The respiratory device comprises a respirator that is or can be linked with an endotracheal tube or a respiratory mask. The respiratory device is provided with a control/regulation unit (1) for controlling and checking the expiration phase and with actuators (2, 39) controlled by the unit for actively influencing expiration and producing any expiration pattern during the expiration phase.

### BACKGROUND OF THE INVENTION

1. Field of the Invention

The invention relates to an equipment for mechanical ventilation which is used to ventilate a patient, comprising at least a ventilator and an endotracheal tube or a ventilatory mask. In addition the invention relates to a method to ventilate a patient, in which operating parameters are measured during mechanical ventilation which are used to control ventilation.

2. Description of the Prior Art

Artificial or mechanical ventilation takes place either in the controlled mode or in a mode of (supported) spontaneous breathing. In the first case the ventilator has complete control over the respiratory pattern, whereas in the second case the—at least—partially spontaneously breathing patient has a considerable influence on the respiratory pattern. However, a feature which is common to all modes of mechanical ventilation is that the ventilator exclusively exerts influence on the inspiratory phase. The ventilator takes over the mechanical work of breathing exclusively during the inspiratory phase. The expiration—from the perspective of the ventilator—occurs passively, that is the energy stored in the elastic tissue elements of lung and thorax generates the drive for expiration. Consequently, the passive deflation of the lung follows an exponential decay curve with a time-constant which is determined by the volume distensibility (compliance) of the respiratory system as well as by the sum of the airway resistances of the biological and artificial airways (Guttmann J, Eberhard L, Fabry B, Bertschmann W, Zeravik J, Adolph M, Eckart J, Wolff G. Time ConstantNolume Relationship of Passive Expiration in Mechanically Ventilated ARDS Patients. Eur Respir J 8(1):114-20, 1995).

On the part of the ventilator only the end-expiratory pressure level (PEEP) and the expiratory time which is available is actively influenced up to now.

A technical realization is already known in which the patient is relieved from the flow-dependent airflow resistance of the endotracheal tube. This mode of support is called ATC (Automatic Tube Compensation) (Fabry B, Guttmann J, Eberhard L, Wolff G. Automatic compensation of endotracheal tube resistance in spontaneously breathing patients. Technol Health Care 1: 281-291, 1994). (ATC: registered trademark (Dräger Medical, Lübeck, Germany)

In German Patent 101 31 653 C2 a method and a device for supply of respiratory gas to a person is proposed. In the context of sleep related breathing disorders preferentially in the frame of homecare ventilation, the airway pressure at the breathing mask can selectively be set either lower or higher than the level of ambient pressure. By decreasing the airway pressure below ambient pressure, the need of mechanical stabilization of the upper airways by overpressure can be determined. Furthermore a screening of snoring syndromes as well as of the susceptibility of obstruction in asthma is possible. In the frame of respiratory therapy, this method can also be applied to reduce the airway pressure below ambient pressure during the expiratory cycles.

From German Patent 195 16 536 C2 a method and a ventilator are known in which the advantages of pressure-controlled ventilation by controlling the airway pressure, and the volume-controlled ventilation by controlling the respiratory volume and the free (Durchatembarkeit) should be combined. By stepwise adaptation of the inspiratory pressure level, a pressure-controlled ventilation with an adjustable tidal volume can be applied. There are setpoints allowed for the inspiratory and expiratory airway pressure, which produce switching from one respiratory phase to the other in the case they are exceeded due to active inspiratory or expiratory efforts.

From WO 02/082997 A2 a control device to preset an airway pressure level is known. Using this device should allow determination of those characteristics of airway pressure which are advantageous with respect to the momentary physiological status of the patient. The setting of airway pressure is realized in dependence of automatically detected respiratory events like apneas or hypopneas. Accordingly, the therapeutic airway pressures are adapted.

### SUMMARY OF THE INVENTION

The present invention provides a ventilatory equipment that allows advanced diagnostics including the analysis of respiratory mechanics of the respiratory system (lung and thorax) and an advanced therapy with respect to practically all indications of artificial ventilation are possible.

The ventilatory equipment of the invention controls the expiratory phase with controllable actuators for providing active manipulation of the expiration and for generation of a user-defined pattern of expiration during the expiratory phase.

In particular the expiratory patterns can be generated by forced time-dependent courses of airflow and/or of airway pressure and/or of respiratory gas volume.

Preferentially a controller unit is provided to force an expiratory pattern for an expiratory phase, whereby a measuring device which is connected with the control unit records the course of expiration during an expiratory phase at the natural exhalation of the patient as well as means for limitation or acceleration of the patient' expiration are provided which are connected to the control unit.

According to the present invention, one expiratory pattern is predefined for one expiratory phase at a time and the natural exhalation of the patient is adapted at the predefined expiratory pattern either by limitation or by acceleration of the exhalation during the expiratory phase.

For registration of the expiratory pattern during natural exhalation of the patient, the airway pressure and/or the airflow rate and/or the gas volume are measured during the expiratory phase and are compared with the corresponding data of the predefined expiratory pattern and the actual expiration is affected.

According to the present invention the respiratory pattern (airflow, airway pressure and breathing volume) during the expiratory phase follows a certain time course. Consequently an active control of the respiratory pattern is introduced particularly by a change of the courses of airway pressure and airflow rate during the expiratory phase.

The method can be applied during controlled ventilation as well as during spontaneous breathing both for diagnostics and therapeutic purposes.

For example in patients with obstructive ventilatory disorders, the airways can be mechanically stabilized by setting a higher airway pressure during a high expiratory flow compared to the pressure at a lower expiratory flow (imitation of the expiratory flow limitation due to purse one's lips). In patients with acute pulmonary distress, the formation of atelectases and the concomitant occurrence of ventilator induced lung injury (VILI) can be reduced by a specific limitation of the expiratory flow. The latter is realized by a reduction of the effective shear forces.

An active manipulation of the respiratory pattern during the expiratory phase is notedly reasonable and desirable with regard to diagnostics as well as to therapy.

In a preferential embodiment, the control unit with its sensors and with the actuators of the ventilation equipment comprises a functional unit. The functional unit can be implemented in an existing ventilator or can be connected with a ventilator as an external device.

To implement this functional unit into a medical device the technology of modern ventilators can be utilized because in principle an active manipulation of the expiratory pattern is already possible. The expiratory valve can perform the function of reducing the expiratory flow. If required, a supplementary subathmospheric pressure source could be implemented into the ventilator.

If realized as a separate unit, the elements (actuators) influencing the pneumatic system can be attached directly to the expiratory connector of the ventilator.

Furthermore, an upgrade of hardware and/or software of existing ventilators can be realized in an advantageous way.

Finally the realization as an external device allows an extension of functionality in already existing older ventilators.

A preferential design of the invention provides sensor inputs in the control unit to allow for a closed-loop control based on pressure and/or flow- and/or volume sensors, that is using measured respiratory data. Optionally already one measurement category may be sufficient to adjust the desired expiratory pattern. However, it is particularly advantageous with respect to patient safety to typically consider the airway pressure in the control loop. The combination of several sensory inputs results in an advantageous improvement of the precision of control.

According to an alternative design, the control unit may contain inputs for anthropometrical or physiological data. Inputs for anthropometric data allow in an advantageous manner an automatically adapted ventilatory setting for example according to height and weight of the patient. Inputs for physiologic data typically but not exclusively include informations about the illness or about the actual status of the patient's illness. By using inputs for such types of data, the control system can be advantageously adapted to the individual disease pattern.

Advantageously—especially when the control unit is realized as an external device—this unit influencing the respiratory airflow course is realized according to the principle of a controller with fixed setpoints. The desired expiratory pattern can be simply realized by a fixed mechanical coupling of typically a volume pump (without controller).

In all other cases it is advantageous if a closed-loop control is used with sensor inputs—typically but not exclusively—respiratory measuring data like pressure, flow and volume. By using respiratory measuring data, the safety of the method can be improved in an advantageous manner. For example—but not exclusively, by considering the airway pressure, short-term pressure peaks due to coughing or pressing can be avoided. By incorporation of non-respiratory measured data, the influence of the expiratory pattern, for example on the cardiovascular system, can be considered in an advantageous manner.

If applicable, the manipulation of the respiratory airflow course can be realized according to the principle of a controller with fixed setpoints. This type of manipulation can be selected in an advantageous manner particularly in the case where the control-loop of the ventilation equipment cannot react fast enough to realize the desired manipulation.

A complementary design of the invention provides that either the airway pressure or the flow rate or the volume during the expiratory phase are controlled typically as a function of time and/or of pressure and/or of flow and/or of volume. This type of control can be particularly selected in an advantageous manner, when the changes of expiration should be realized in dependence of the respiratory mechanics of the diseased lung.

Optionally the manipulation of the expiration is realized in dependence or in independence of the respiratory pattern during inspiration and of the ventilatory type. In this way, it can be achieved in an advantageous manner, that—depending on the wish of the user—either the expiratory pattern is exclusively set (independent manipulation) or a simplified combination mode (dependent manipulation) can be selected.

According to another embodiment of the invention the manipulation of the expiration can be applied during controlled mechanical ventilation, during supported or during non-supported spontaneous breathing.

Thereby the manipulation (influence) of the expiration can be achieved in an advantageous manner for every possible application of respiratory therapy and the active manipulation of the expiration can be combined respectively with every ventilatory type and mode in an advantageous manner.

The manipulation of the expiration can be applied in an advantageous manner during endotracheal intubation or during ventilation via a breathing mask. Consequently, the manipulation of the expiration can be applied independently from the access to the airways. In addition the influence of the access to the airways on the expiratory pattern can be considered.

Advantageously the shape/course of the expiratory function can be arbitrarily, it can be for example a simple ramp or a half-sine-wave. Good approximations towards complex control functions with a physiological rationale can be achieved in an advantageous manner with technically simply realizable functions—typically when using a controller with fixed setpoints—.

Optionally the expiratory function is combined with positive end-expiratory pressure (PEEP) or replaces the latter. The active manipulation of the expiratory pattern can be combined with the set PEEP, without changing it. In an advantageous manner the expiratory function can be designed such that it replaces the PEEP or it takes over the role of PEEP.

According to an embodiment of the invention, the change of pressure, of flow or of volume, which is effected by the control unit compared to a passive expiration, can have a positive or a negative sign or also changing signs.

The limitation of the expiratory flow causes an increase of the mean pulmonary volume during the expiration, which has a mechanically stabilizing effect on the diseased lung. Particularly at a short expiratory time an airflow acceleration which follows an airflow limitation can keep constant the expiratory volume in an advantageous manner and can avoid an overinflation (intrinsic PEEP) of the lungs.

If appropriate the duration of the controlling phase can be variable. The period of active control of the expiration may be independent from the duration of the expiratory phase (typically shorter). Thereby the period of control is determined exclusively by the clinical demands.

In addition there exists the alternative that the duration of the controlling phase exceeds the duration of a single expiration. Advantageously the manipulation of the expiratory pattern can be realized over a variable number of breaths according to a presetting “A”, than can be inactivated or can be continued according to a new presetting “B” in terms of polymorphous ventilation.

The shape/course of the expiratory function can act in accordance with the special application and with the goals which are to be achieved by using the controlling technique. The high variability of the controlling technique guarantees that the expiratory pattern can be approximated on the individual patient as well as on the relative demands of the treating physician for example with respect to the analysis of the respiratory mechanics during expiration.

It is advantageous if the shape/course of the expiratory function is particularly adapted during the controlling period. By this means—dependent from the clinial demands—the presettings for controlling the expiratory pattern can be changed within a breath (intratidal) or breath-by-breath.

Advantageously, the settings of the controller can be realized manually or automatically, particularly in an adaptive way. The advantageous plasticity in the application of the method enables that the physician can pursuit typically short-term goals, or the physician can declare goals with the system, which the system tries to reach within a selectable period of time.

As the case may be, several functions can be superimposed or can alternate. Thereby an adaptation to fast or slow properties of respiratory mechanics (time constants) of the respiratory system is possible.

The period of time of the expiration can either be given by the ventilator or by the patient or by a combination of both. Advantageously the system recommends presettings for the expiratory time.

If required the expiratory time can be prolonged or shortened. Consequently, the system considers in an advantageous manner accomplished changes of the expiratory time.

Advantageously, parameters of respiratory mechanics are measured such as for example resistance, compliance or expiratory flow limitation. Advantageously the variables pressure, flow and volume can be interconnected in terms of a complex controlling.

Further advantageous designs of the invention are described in the other subclaims.

### BRIEF DESCRIPTION OF THE DRAWINGS

In the following the invention is explained in detail by means of figures.

It shows:

FIG. 1 is a schematic illustration of a functional unit according to the present invention including a control unit as well as actuators;

FIG. 2a to 2d are different pressure-volume-diagrams;

FIG. 3 shows flow, pressure and volume curves during inspiration and expiration;

FIG. 4 is a schematic illustration of alveoli in the collapsed status;

FIG. 5 is a schematic illustration of alveoli in the native status;

FIG. 6 is a diagram showing the dynamic pressure-volume-loop of a breath; and

FIG. 7 is a diagram showing the expiratory flow-time-curve of a breath.

### DETAILED DESCRIPTION OF THE INVENTION

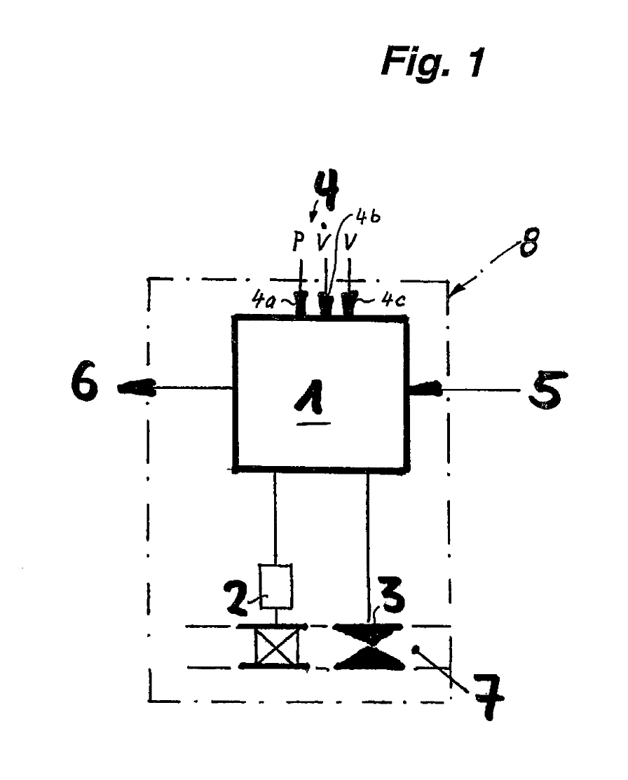

FIG. 1 schematically shows ventilation equipment WITH a functional unit 8 with three main components, namely a preferentially electronic control unit 1 and as actuators a controllable electromechanical unit 3 for changing the airflow resistance and a controllable unit 2 for changing the expiratory pressure.

The control unit 1 includes signal inputs 4 for pressure signals 4a, flow signals 4b and volume signals 4c as well as a signal input for the setpoint input 5 for the desired expiratory breathing pattern. The control unit 1 provides control signals to both actuators 2 and 3 as well as via the output 6 to the expiration controller of the ventilator.

The control unit 1 can be set up in combination with the sensors which are connected with the inputs and with the actuators a functional unit. With respect to the connection of the complete functional unit with the ventilator, there are in principle two types of realization possible. On the one hand an implementation into a ventilator is possible. The technology of modern ventilators enables in principle an active manipulation of the expiratory pattern. Here the expiratory valve can take over the role of limiting the expiratory flow and in addition a source of subathmospheric pressure 2 can be implemented into the ventilator if required. On the other hand a separate functional unit can be utilized, whereby then the actuators are directly connected with the expiratory connector 7 of the ventilator.

As already mentioned, an active manipulation of the respiratory pattern during the expiration phase is most reasonable and desirable with respect to diagnostics as well as to therapy. For this some examples are given in the following:

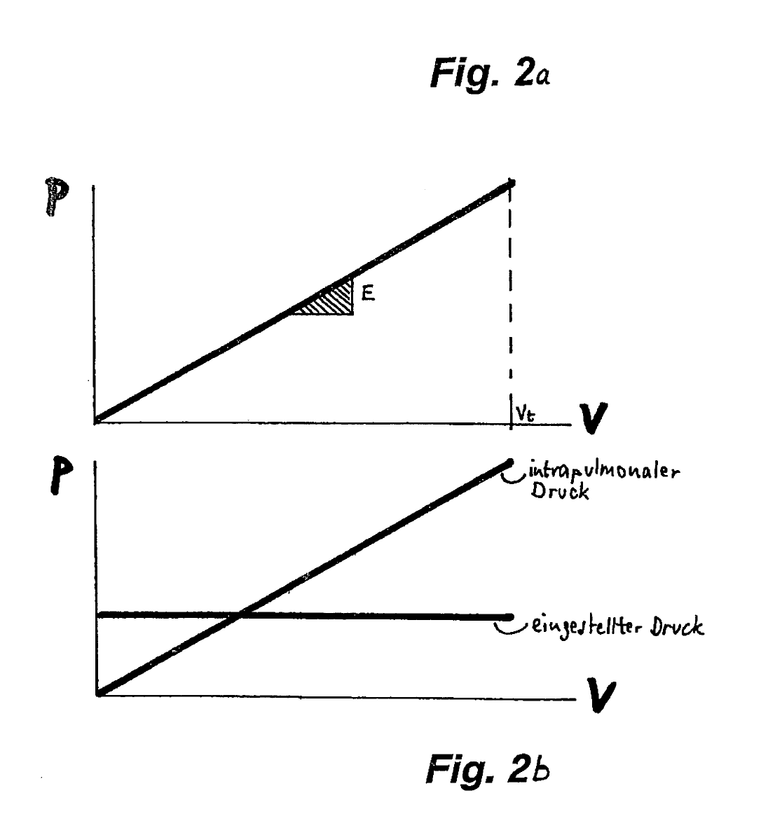

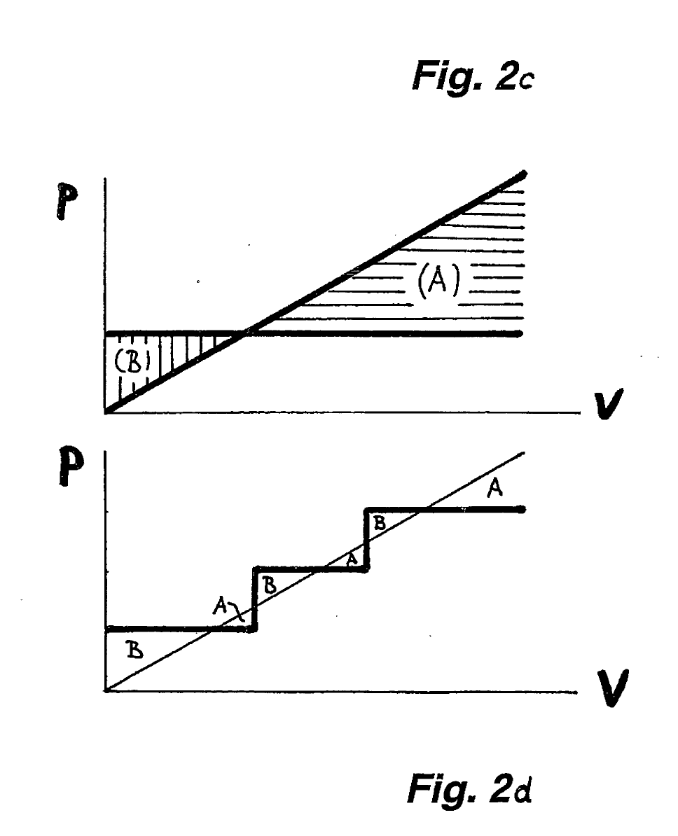

Diagnostics: It is known that the mechanical properties of the respiratory system differ between inspiration and expiration. One of the reasons is a phenomenon called intratidal alveolar recruitment. In other words: there is alveolar tissue recruited during inspiration that collapses in the following expiration. It is expected that the difference between inspiratory and expiratory respiratory mechanics allows a quantification of the amount of intratidal recruitment/derecruitment. Hence, there is a considerable interest on the part of the intensive care doctors to analyze respiratory mechanics of lungs of the critically ill separately in inspiration and expiration (respiratory monitoring). This differentiation has failed up to now due to the nonlinear flow pattern of the expiratory phase. The lung is - in the mechanical sense - a passive elastic body with a more or less linear relationship between pressure and volume as this is shown in FIG. 2a. The slope of the pressure-volume curve equals the Elastance E (=1/Compliance). As volume continuously changes during expiration—volume decreases from the tidal volume (VT)—the driving pressure for expiration decreases at the same time. The consequence is an exponential shape of the expiratory flow-time curve. (FIG. 7). The concurrent change of gas flow and volume makes the differential equation that describes the mechanical properties of the respiratory system (equation of motion) insolvable. A distinct solution would be possible, however, if the flow would be (as an example) constant during the whole expiration. The latter is the case when the driving pressure would be steady (or not volume dependent) during the whole expiration (compare FIG. 2b). For this case, two areas are to be distinguished (compare FIG. 2c):

(A) the intrapulmonary pressure is above the set pressure; and
(B) the intrapulmonary pressure is below the set pressure.
For the area (A) this means that the “elastic” pressure of the lung would generate a higher expiratory flow than the flow that is given by the set pressure difference. In this case expiratory flow has to be “slowed down”. This could be reached exemplarily by increasing the flow resistance by actuator 3 (FIG. 1). For the area (B) the intrapulmonary pressure obviously is not sufficient to generate an expiratory flow as is expected by the set pressure difference. In this case a flow increase is necessary; this can be realized, for example, by adding a regulated negative pressure source 2 (FIG. 1). Generally spoken, the expiratory flow has to be reduced any time a situation (A) is desired, and the expiratory flow has to be increased any time a situation (B) is desired. To emphasize this, FIG. 2d shows another example with which the Exspiration should be realized by three phases of steady flow.

A specific application for the diagnostic use is the analysis of nonlinear, dynamic respiratory mechanics. In the critically ill, I the mechanical properties of the lung (elasticity and resistance) are not constant, but they change even within the taking of a breath. The variability of respiratory mechanics manifests in many patients in a considerable intra-breath non-linearity.

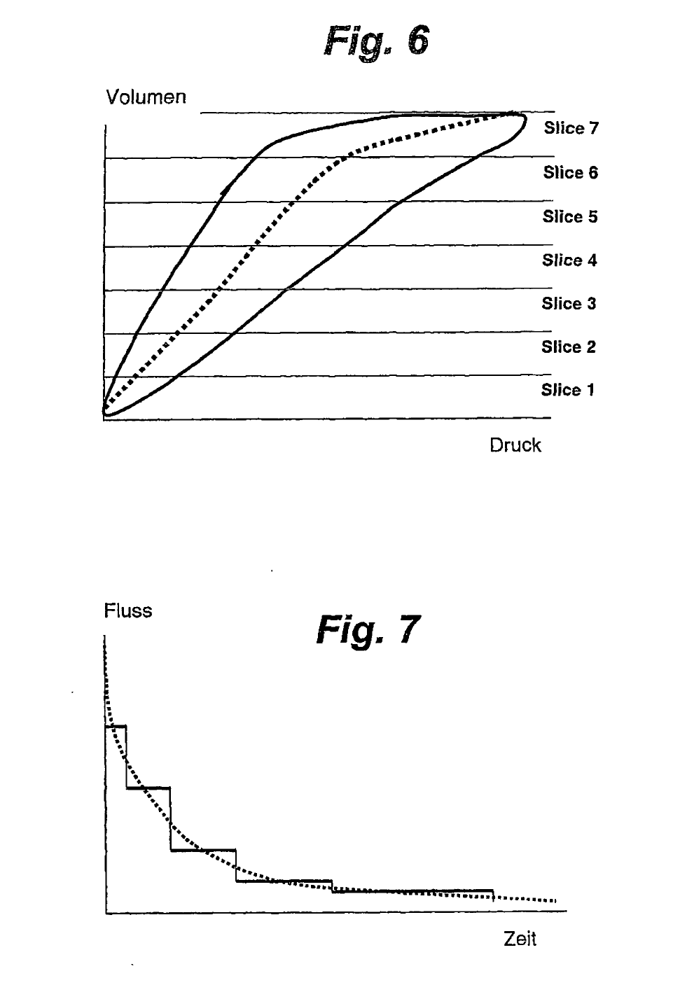

FIG. 6 schematically shows the dynamic pressure-volume-loop of a breath during controlled mechanical ventilation. The change in slope of the dynamic PV-loop expresses the nonlinearity of the compliance, the different width of the PV-loop expresses the nonlinearity of flow resistance. New diagnostic procedures permit the analysis of nonlinear respiratory mechanics within the breath. To do so, the PV-loop is divided into several volume segments of equal size (SLICES) (FIG. 6) and respiratory mechanics are analyzed for each segment separately using a mathematic procedure (Guttmann J, Eberhard L, Fabry B, Zapping D, Bernhard H, Lichtwarck-Aschoff M, Adolph M, Wolff G. Determination of Volume-Dependent Repiratory System Mechanics in Mechanically Ventilated Patients Using the Ne SLICE Method. Technol Health Care 2: 175-191, 1994).

It was not possible to date to perform the analysis of respiratory mechanics separately for inspiration and expiration. To stabilize the algorithm, in- and expiratory data had to be included into the analysis. According to the present invention, however, the gas flow during expiration could be set segment-wise constant.

FIG. 7 shows an expiratory flow-time curve of a breath. The dotted line shows the natural, exponential shape of the flow curve. To the exponential flow curve, a stair-shaped flow curve is adapted, the length of single steps being different. The solid line in FIG. 7 shows such a realization of an adapted stepwise liberalized expiratory flow. The different durations of the phases with constant flow correlate with the SLICE volume (compare FIG. 6). Therefore algorithmic stability is given and a separate analysis in inspiration and expiration is possible. In principle, according to the invention, any expiratory flow pattern and pressure pattern may be realized. This includes increasing and decreasing ramps with variable slope, proportionality to time, volume and flow as well as any nonlinear functions as sine or sawtooth or others.

Therapeutic use: In Patients with an obstructive disease, collapse of small airways during expiration is a common phenomenon. This mechanism not only causes increased work of breathing and under-ventilation of the lung. The impediment of expiration leads to an increase of intrathoracic pressure (dynamic hyperinflation) with serious consequences for the hemodynamic stability up to severe shock. An active change of expiratory flow in terms of a slowing down could correct the pathomechanism by splinting the airway.

In patients with acute or chronic respiratory failure, mechanical ventilation promotes additional damage to the already injured lung (ventilator associated lung injury-VALI). Above all, the shear forces that are induced by repeated closure of alveoli during expiration and their reopening during early inspiration have been linked to VALI (atelectrauma). Up to now, only by setting a constant end-expiratory pressure (PEEP) was used to influence the global strain within the lung. An active change of the expiratory flow pattern (in terms of slowing down the expiratory flow) might selectively stabilize instable alveoli. By active circumvention of high expiratory flows the global shear forces within the lungs could be reduced and VALI could be prevented.

On the other hand, the disturbed gas exchange in these patients obliges the caregiver to increase the breathing frequency thereby reducing the expiratory time. As a result expiration may become incomplete and increased intrapulmonary pressure may occur (intrinsic PEEP). A controlled increase of expiratory flow could remove PEEPi in this situation.

The artificial airways (endotracheal tube, tracheal cannula) prevent the natural cough and expectoration in ventilated patients. On one hand the tube is the major barrier for bronchial secretions and it prevents the glottic closure and tracheal collapse during coughing. In addition, the patients cough is reduced by sedatives and opioids. A specific manipulation (for example, biphasic) of the expiratory flow the transport of secretions and expectoration might be notably improved.

Patients that need mechanical ventilation have a high demand of sedatives. It has been proven that survival is negatively correlated with the amount of administered sedatives. Sedatives are needed, because mechanical ventilation is felt to be extremely unpleasant by the patient. It is known that the ventilation mode during inspiration influences patient comfort. During spontaneous breathing, the decrease of inspiratory muscle activity controls expiratory flow. In contrast, no such mechanism is available during mechanical ventilation. Imitation of a natural breathing pattern (by a specific presetting of the ventilator) would significantly increase patient comfort.

The severely ill lung is characterized by mechanical nonlinearity and by mechanical inhomogeneity. Active expiratory control will lead to a more homogeneous ventilation as possible to date with passive expiration. The latter includes the variation of expiratory control on y breath-by-breath base (polymorphous ventilation).

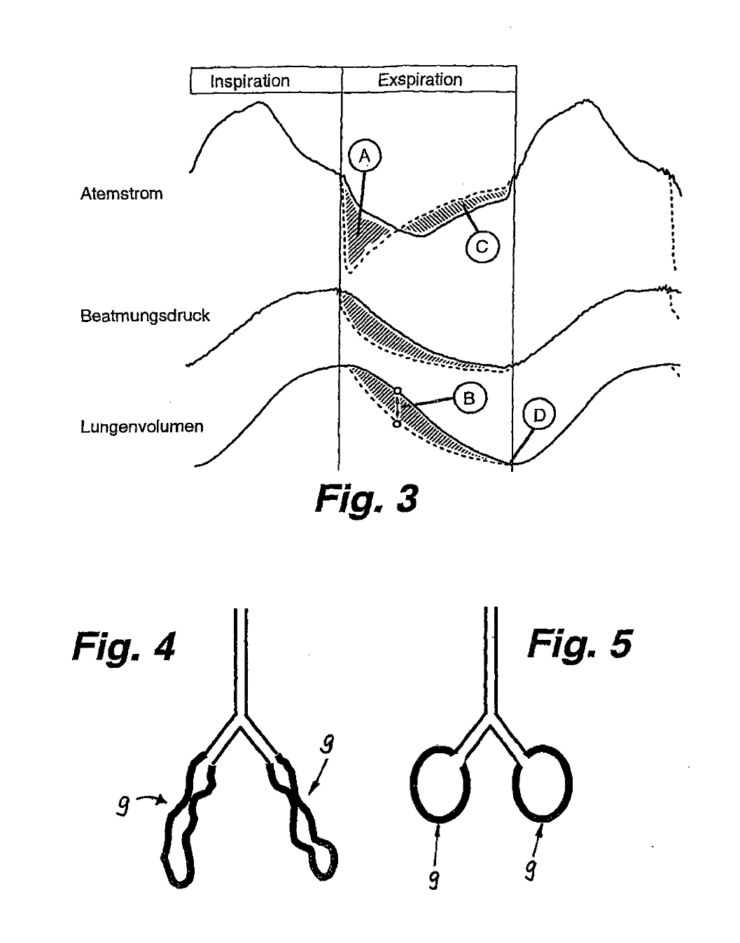

FIG. 3 shows a scheme for the therapeutic use of active expiratory control. In the example shown, the dotted lines show the natural course of passive expiration. The course is accomplished as from the beginning of passive expiration the pressure difference between alveoli and atmospheric pressure is decreasing. Therefore alveolar pressure which causes the peak flow at the beginning of the expiratory phase decreases quickly after onset of expiration. The risk of collapse of alveoli (9) is increased in the early phase of expiration due to the high transmural pressures. The injured lung is at high risk due to the formation of atelectasis. Cyclic collapse and reopening of alveoli (9) induces irreversible damage of the lung tissue. FIG. 5 shows alveoli (9) in their native situation.

Due to active expiratory control (FIG. 3 solid line) gas is retained within the lung during the first half of expiration as compared to passive expiration (dotted line). Therefore the lung is mechanically stabilized and the injurious alveolar collapse is reduced. Early in expiration flow is markedly reduced (A). As, compared to passive expiration, less air is being exhaled in this phase, the intrapulmonary gas volume in higher (B). Because the flow rate is increased at the end of expiration (C), the same volume is exhaled during the complete expiration. Alveolar collapse is prevented, because in the second half of expiration the transalveolar pressure gradient is reduced as compared to the first half of the expiration phase. In both cases, the end-expiratory volume is the same (D). As the schematic illustration shows, it is possible to implement a biphasic modification of expiration without reducing the pressure below the set positive end-expiratory pressure (PEEP).

1-29. (canceled)

30. A respiratory device for use in ventilation of a patient comprising:
a ventilator for connection or connected to an endotracheal tube or to a respiratory mask and expiration phase controllable actuators for providing regulation and control for actively influencing expiration and for generating an arbitrary expiration pattern during expiration.

31. A device in accordance with claim 30 wherein:
the arbitrary expiration pattern is generated by at least one of time-dependent airflow courses, airway pressure courses and respiratory gas volume changes, wherein each of the courses and changes may be preset.

32. A device in accordance with claim 30 comprising:
a control and regulation unit for presetting the arbitrary expiration pattern during expiration, a measuring device, for assessing the expiration during natural expiration of the patient, which is connected with the actuators, means for limiting expiration of the patient and means for accelerating expiration of the patient.

33. A device in accordance with claim 32 wherein:
the control and regulation unit includes sensor inputs for at least one pressure and flow and/or volume sensors are provided for a closed-loop control for measuring respiratory data.

34. A device in accordance with claim 32 wherein:
the control and regulation unit includes inputs of anthropometrical or physiological data.

35. A device in accordance with claim 32 wherein:
the control and regulation unit includes a controller with set presettings.

36. A device in accordance with claim 33 comprising:
a functional unit including the control and regulation unit, the sensors and the actuators.

37. A device in accordance with claim 36 wherein:
the functional unit comprises an existing ventilator.

38. A device in accordance with claim 36 wherein:
the functional unit comprises an external device connectable with a ventilator.

39. A method of controlled and regulated mechanical ventilation of a patient comprising:
measuring, during mechanical ventilation, operational parameters, and controlling the ventilation with the measured operational parameter; and wherein
an expiration pattern is preset for at least one expiration phase and natural expiration of the patient is adapted to the preset expiration pattern by limiting or by accelerating the expiration during the expiration phase.

40. A method in accordance with claim 39 wherein:
at least one measurement of airway pressure, airflow and respiratory gas volume is made during the expiration phase, is compared with the preset expiration pattern and actual expiration is influenced by the at least one measurement.

41. A method in accordance with claim 39 wherein:
the regulated mechanical ventilation is controlled by a closed loop responsive to sensor inputs.

42. A method in accordance with claim 39 wherein:
a respiratory airflow-course is controlled with fixed presettings.

43. A method in accordance with claim 40 wherein:
one of pressure, flow and volume during the at least one expiration phase are controlled.

44. A method in accordance with claim 39 wherein:
the expiration phase is dependent upon a breathing pattern during inspiration and ventilation type.

45. A method in accordance with claim 39 wherein:
influencing of at least one expiration phase occurs during controlled mechanical ventilation and during supported breathing.

46. A method in accordance with claim 39 wherein:
the influencing expiration phase occurs during one of endotracheal intubation and ventilation by a breathing mask.

47. A method in accordance with claim 39 wherein:
a pattern of the expiration phase is an arbitrary function.

48. A method in accordance with claim 47 wherein:
the arbitrary function is either combined with the positive end-expiratory pressure (PEEP) or the arbitrary function replaces PEEP.

49. A method in accordance with claim 44 wherein:
changes of the pressure, the flow or the volume have one of a positive, or a negative sign or alternating signs in comparison to a passive expiration.

50. A method in accordance with claim 41 wherein:
the control and regulation is performed with a variable duration.

51. A method in accordance with claim 41 wherein:
the control and regulation is longer in duration than a duration of a single expiration phase.

52. A method in accordance with claim 48 wherein:
the arbitrary function is provided by a control of the mechanical ventilator.

53. A method in accordance with claim 48 wherein:
the arbitrary function is adapted during operation of the mechanical ventilator.

54. A method in accordance with claim 41 wherein:
settings and adjustments of the control and regulation are performed in an adaptive way.

55. A method in accordance with claim 44 wherein:
functions in combination with each other or alternatively control the expiration phase.

56. A method in accordance with claim 39 wherein:
a time period of expiration is preset either by the ventilator, the patient or a combination of both.

57. A method in accordance with claim 44 wherein:
a time of the expiration phase is shortened or lengthened as required by the patent.

58. A method in accordance with claim 39 wherein:
parameters of respiratory mechanics are measured.

59. A method in accordance with claim 44 wherein:
the control and regulation is performed with a variable duration.

60. A method in accordance with claim 44 wherein:
the control and regulation are longer in duration than a duration of a single expiration.

61. A method in accordance with claim 43 wherein:
settings and adjustments of the control and regulation are made by one of manual adjustment or automatic adjustment in an adaptive manner.

62. A method in accordance with claim 44 wherein:
the control is a function of at least one of time, pressure, flow and volume.

63. A method in accordance with claim 48 wherein:
the arbitrary function is one of a ramp, a staircase or half a sine wave.

64. A method in accordance with claim 59 wherein:
the parameters comprise at least one of resistance, compliance or expiratory flow limitation.
